(19) United States
(12) Patent Application Publication
(10) Pub. No.: US 2016/0135826 A1
(43) Pub. Date: May 19, 2016

(54) FLUID SUPPLY AND SUCTION DEVICE

(71) Applicant: David Michael Chadbourne, Amherst, MA (US)

(72) Inventor: David Michael Chadbourne, Amherst, MA (US)

(21) Appl. No.: 14/546,666

(22) Filed: Nov. 18, 2014

(51) Int. Cl. A61B 17/22; A61M 1/00

(52) U.S. Cl. CPC A61B 17/22; A61M 1/0058; A61M 1/0035; A61B 2017/22079; A61B 2017/22082

(57) ABSTRACT

A fluid supply and suction device is provided and includes a tube, a container including first and second parts, which are sealably attachable to define a container interior, the first part being formed to define first and second apertures and being coupled with the tube such that the tube is fluidly communicative with the container interior and a valve system coupled with the first part and configured to admit via the first aperture fluid into the tube such that the fluid flows through the tube and to suction via the second aperture fluid and fragmented particles through the tube and into the container interior.

### BACKGROUND OF THE INVENTION

The subject matter disclosed herein relates to a fluid supply and suction device and, more particularly, to a fluid supply and suction device that can be used to expedite certain medical procedures including, but not limited to, percutaneous nephrolithotomy (PCNL).

Various surgical procedures have been developed to remove objects or debris from a body. Percutaneous nephrolithotomy (PCNL) is one example of such a surgical procedure and serves to remove stones from a kidney by way of a small puncture wound (up to about 1 cm) through the skin. During PCNL, a retrograde pyelogram (RP) or a CT scan is used to locate the stone in the kidney at which point an incision is made in the loin (or, alternatively, between the ribs). A percutaneous nephrolithotomy (PCN) needle is then passed through the puncture wound and into the pelvis of the kidney with a position of the needle confirmed by fluoroscopy. A guide wire is passed through the needle into the pelvis and the needle is withdrawn with the guide wire still inside the pelvis. Dilators or balloons are then passed over the guide wire and a working sheath is introduced.

During the procedure, a stone that is too large to be removed from the body via the incision and the sheath is fragmented or demolished. In the case of fragmentation, the stone may have an initial diameter of 1-2 cm (or, generally, larger) and is broken down into smaller stones of 4-7 mm sizes. These smaller stone fragments are then removed from the body by hand-held tools in a one-by-one sequence over the course of an extended period of time. In the case of the stone being demolished, the stone is broken down into dust sized particles that can be fairly easily flushed out of the body but the time taken to break down the original stone into dust is, again, time consuming. In both cases, the length of time of the process is extended and can be extremely costly.

### BRIEF DESCRIPTION OF THE INVENTION

According to one aspect of the invention, a fluid supply and suction device is provided and includes a tube, a container including first and second parts, which are sealably attachable to define a container interior, the first part being formed to define first and second apertures and being coupled with the tube such that the tube is fluidly communicative with the container interior and a valve system coupled with the first part and configured to admit via the first aperture fluid into the tube such that the fluid flows through the tube and to suction via the second aperture fluid and fragmented particles through the tube and into the container interior.

According to another aspect of the invention, a fluid supply and suction device, is provided and includes a tube having a first tube end, which is removably insertible into a sheath terminating in a percutaneous cavity, and a second tube end, a container including first and second parts, which are sealably attachable to define a container interior, the first part being formed to define first and second apertures and being coupled with the second tube end such that the second tube end is fluidly communicative with the container interior and a valve system coupled with the first part and configured to admit via the first aperture fluid into the second tube end such that the fluid flows through the first tube end and into the percutaneous cavity and to suction, via the second aperture, the fluid and fragmented particles from the percutaneous cavity and into the container interior.

According to yet another aspect of the invention, a method of operating a fluid supply and suction device is provided and includes removably inserting a tube into a sheath terminating in a percutaneous cavity, sealably attaching first and second parts of a container to define a container interior, coupling the tube with the first part such that the tube is fluidly communicative with the container interior, admitting fluid into the tube such that the fluid flows through the first tube end and into the percutaneous cavity and suctioning the fluid and fragmented particles from the percutaneous cavity and into the container interior.

These and other advantages and features will become more apparent from the following description taken in conjunction with the drawings.

### BRIEF DESCRIPTION OF THE DRAWINGS

The subject matter, which is regarded as the invention, is particularly pointed out and distinctly claimed in the claims at the conclusion of the specification. The foregoing and other features, and advantages of the invention are apparent from the following detailed description taken in conjunction with the accompanying drawings in which:

FIG. 1 is a side sectional view of a fluid supply and suction device in accordance with embodiments;

FIG. 2 is an enlarged view of a portion of the fluid supply and suction device of FIG. 1;

FIG. 3 is an enlarged side schematic view of another portion of the fluid supply and suction device of FIG. 1;

FIG. 4 is a perspective view of a container of the fluid supply and suction device of FIGS. 1-3;

FIG. 5 is a flow diagram illustrating a method of operating a fluid supply and suction device in accordance with embodiments; and

FIGS. 6A and 6B are schematic diagrams illustrating an additional embodiment of the fluid supply and suction device of FIGS. 1-4.

The detailed description explains embodiments of the invention, together with advantages and features, by way of example with reference to the drawings.

### DETAILED DESCRIPTION OF THE INVENTION

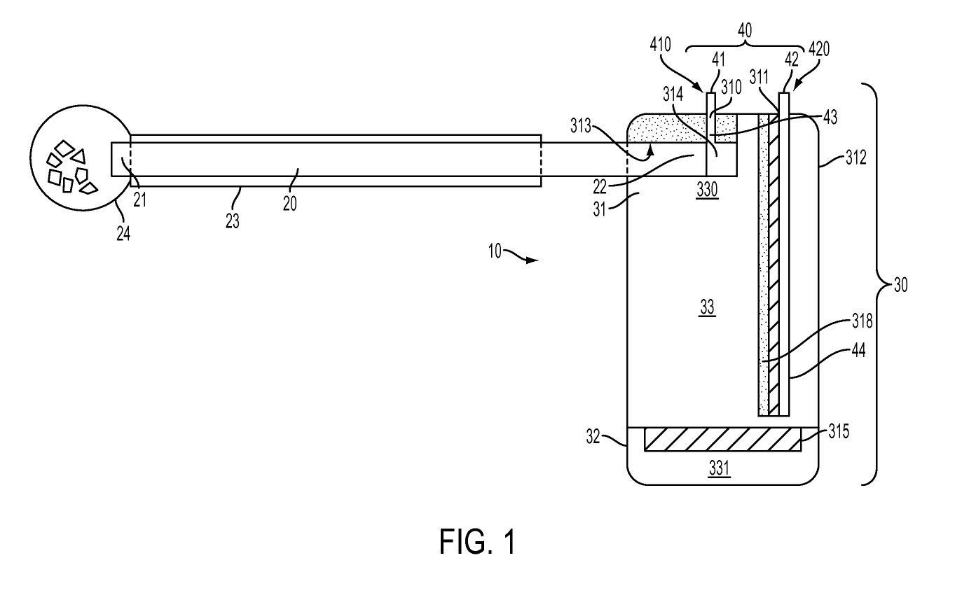

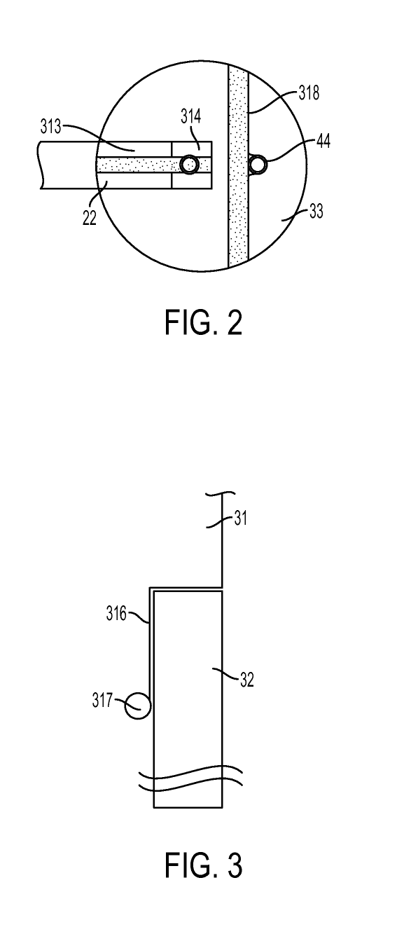

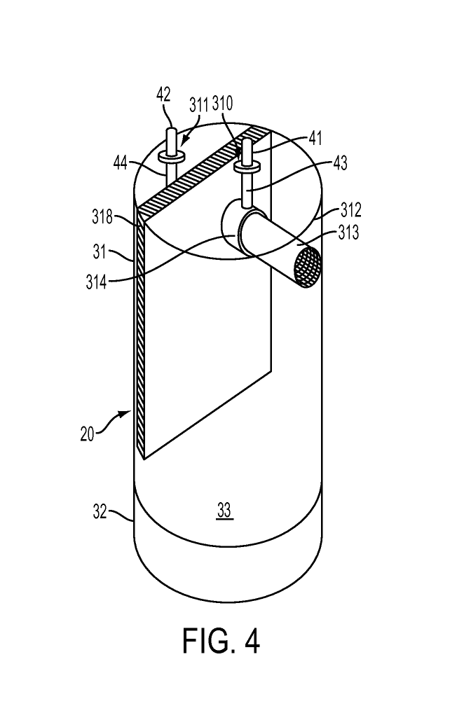

With reference to FIGS. 1-4, while PCNL may be suitable to remove stones of up to or more than 2 cm in size and this or other procedures are available to remove dust formed from stones that have been completed demolished, a fluid supply and suction device 10 (hereinafter referred to as a “device 10”) is provided to facilitate removal of stones that are up to about 4-7 mm in diameter while still removing smaller particles. Such “fragmented” stones or particles are often formed from the fragmentation of a larger stone and would normally have to be removed by hand-held tools during extended and expensive surgical procedures. As will be described below, however, the device 10 allows the fragmented particles to be removed by fluid suction in substantially reduced amounts of time.

As shown in FIGS. 1-4, the device 10 includes a tubular member 20, a container 30 and a valve system 40.

The tubular member 20 has a first tube end 21 and a second tube end 22. The first tube end 21 is removably insertible into a nephroscopy sheath 23 (hereinafter referred to as a “sheath 23”). This sheath 23 terminates in a percutaneous cavity 24 as described above in which a stone or fragmented particles produced from the fragmentation of a larger stone may be contained. The first tube end 21 may extend through a substantial length of the sheath 23 and may terminate proximate to or within the percutaneous cavity 24. The second tube end 22 is opposite the first tube end 21.

The container 30 includes a first part 31 and a second part 32. The second part 32 may be sealably attached to the first part 31 in a direct or an indirect engagement to define a container interior 33. In accordance with embodiments and, with respect to a direction of gravitation, the first part 31 serves to enclose an upper region 330 of the container interior 33 and the second part 32 serves to enclose a lower region 331 of the container interior 33. In addition, the first part 31 is formed to define a first aperture 310 in a first side of the container 30 and a second aperture 311 in a second side of the container 30. Furthermore, the first part 31 may be coupled with the second tube end 22 such that the second tube end 22 is fluidly communicative with the container interior 33.

The valve system 40 is coupled with the first part 31. By way of such coupling, the valve system 40 is configured to admit, via the first aperture 310, fluid into the second tube end 22. This fluid may be provided as water and may be directed to flows through the tubular member 20 and the first tube end 21 and into the percutaneous cavity under little to no pressure other than pressure resulting from gravitation (i.e., the flowing of the fluid from a relatively high elevation to the percutaneous cavity at a relatively low elevation). The valve system 40 is further configured to suction, via the second aperture 311, the fluid and fragmented particles from the percutaneous cavity and into the container interior 33.

In accordance with embodiments, an inner diameter of the sheath 23 may be about 10 mm (30 fr), an outer diameter of the tubular member 20 is about 6-9 mm (18-27 fr) and an inner diameter of the tubular member 20 is about 5-8 mm (15-24 fr). Thus, tubular member 20 can be inserted relatively easily into the sheath 23 with space defined between the tubular member 20 and the sheath 23 for permitting airflow that will allow for the suctioning to be described below. In addition, with these exemplary sizes, the device 10 can be used to remove fragmented particles of up to 4-7 mm in diameter since fragmented particles of this size can easily pass through the tubular member 20. In practice, during an exemplary PCNL procedure, a relatively large stone that cannot be removed directly can be fragmented into fragmented particles of up to 4-7 mm in diameter. This fragmentation can be executed reasonably quickly without the need to completely demolish the stone into dust sized particles (which is a much longer process). Once fragmented, the fragmented particles can be removed from the percutaneous cavity 24 via the tubular member 20 as will be described below in a substantially reduced amount of time.

In accordance with embodiments, the first part 31 includes a body 312 that is formed to define a receiving chamber 313 and an inlet chamber 314. The receiving chamber 313 is tubular in shape and has inward facing sidewalls that are sized to surround or, in some cases, tightly fit around an exterior surface of the second tube end 22 of the tubular member 20. Alternatively, the receiving chamber 313 may be tapered to receive the second tube end 22 or integrally coupled with the second tube end 22. In any case, a seal between the second tube end 22 and the body 312 may be formed to prevent leakage and to permit vacuum generation in the container interior 33. Where unwanted space exists between the second tube end 22 and the inward facing sidewalls of the receiving chamber 313, a sealant may be provided to fill the space and to maintain the seal.

The inlet chamber 314 is coupled with the valve system 40 and configured to direct fluid from the valve system 40 to the tubular member 20. The inlet chamber 314 is adjacent to the receiving chamber 313 and proximate to the first aperture 310 such that fluid and fragmented particles exiting from the second tube end 22 flow into the inlet chamber 314 and then exit from the inlet chamber 314 into the container interior 33. As shown in FIG. 1, an outer diameter of the inlet chamber 314 may be at least as large as a corresponding dimension of the receiving chamber 313 and at least as large as or larger than an inner diameter of the tubular member 20. Thus, as fluid or fragmented particles flow through and exit from the second tube end 22, sidewalls of the inlet chamber 314 do not impede the various flows.

In accordance with alternative embodiments, it is to be understood that the body 312 may not be formed to define the inlet chamber 314. In these cases, the valve system 40 would be coupled to the receiving chamber 313 or to the tubular member 20 directly. In the former case, second tube end 22 of the tubular member 20 may be displaced from the innermost end of the receiving chamber 313 where a coupling with the valve system 40 would be disposed. With this arrangement, fluid would enter the innermost end of the receiving chamber 313 from the valve system 40 and then proceed into the second tube end 22. In the latter case, the tubular member 20 may include a through-hole defined in a sidewall thereof to permit fluid ingress into an interior thereof

In accordance with further embodiments, the first and second parts 31 and 32 may each include complementary threading 315 such that the first and second parts 31 and 32 are threadably engageable to each other. While such engagement is mechanical, it will be understood that threaded engagement of the first and second parts 31 and 32 is not required and that other embodiments of mechanical or frictional interference and engagement are possible. For example, the first and second parts 31 and 32 may be keyed together or, as shown in FIG. 3, the first and second parts 31 and 32 may each include a complementary frictional surface 316. In any case, at least one or both of the first and second parts 31 and 32 may include a seal element 317 interposable between the first and second parts 31 and 32. As shown in FIG. 3, the seal element 317 may be an O-ring, for example.

The first part 31 may also be configured to redirect flows exiting from the inlet chamber 314. To this end, the first part 31 may include a baffle 318 that is disposed to redirect outflow from the second tube end 22 and the inlet chamber 314. The baffle 318 extends downwardly into the container interior 33 from an interior surface of the first part 31 and is positioned generally between the first aperture 310 and the second aperture 311. The baffle 318 may extend substantially or entirely between sidewalls of both the first and second parts 31 and 32 to define opposite sides of the container interior 33. In accordance with embodiments, the baffle 318 extends only partially into the second part 32 such that, when the first and second parts 31 and 32 are attached, a distance between a lower surface of the baffle 318 and an interior surface of a bottom of the second part 32 is defined.

In accordance with additional or alternate embodiments, an outlet of the inlet chamber 314 may be curved toward the container interior 33. This curvature serves to redirect flows exiting from the inlet chamber 314 and may be employed as a standalone component or in concert with the baffle 318.

The valve system 40 includes a first valve element 41, a second valve element 42, a fluid coupling 43 and a straw 44. The first valve element 41 may be coupled to a fluid source 410 and to the second tube end 22 by way of the fluid coupling 43 and the inlet chamber 314. The first valve element 41 may be provided as a plunger- or butterfly-type valve for example and is operable by a user using one hand to be open or closed. In the open condition, the first valve element 41 is configured to selectively permit fluid flow into the second tube end 22 by way of the fluid coupling 43 and the inlet chamber 314. In the closed condition, the first valve element 41 prevents fluid flow.

The second valve element 42 is coupled to a vacuum source 420 and to the container interior 33 by way of the straw 44. The second valve element 42 may be provided as a plunger- or butterfly-type valve for example and is operable by a user using one hand to be open or closed. In the open condition, the second valve element 42 is configured to selectively suction the fluid and the fragmented particles through the tubular member 20 from the percutaneous cavity 24 and into the container interior 33. Once in the container interior 33, the fragmented particles will settle at the lower region 331 of the container interior 33 and the fluid will either pool on top of the fragmented particles or be suctioned from the container interior 33 and through the straw 44. In the closed condition, the second valve element 42 isolates the container interior 33 from the suction source 420.

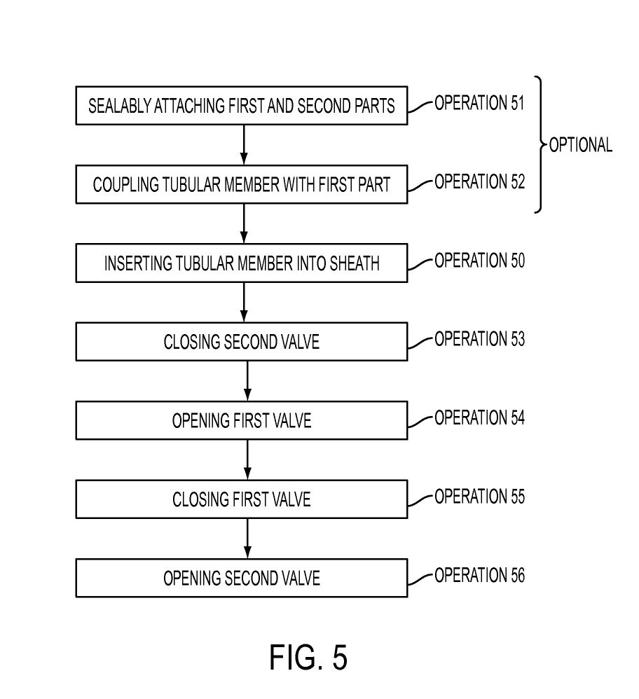

With reference to FIG. 5, a method of operating the device 10 will now be described. This description will be provided with the understanding that the device 10 will be used to remove fragmented stones from a body and that the fragmented stones are up to 4-7 mm diameter and have been formed from the fragmentation of a larger stone or stones in accordance with known methods. The method includes removably inserting the tubular member 20 into the sheath 23 with the sheath 23 terminating at the percutaneous cavity 24 (operation 50). The method also includes sealably attaching the first and second parts 31 and 32 of the container 30 to define the container interior 33 (operation 51) and coupling the tubular member 20 with the first part 31 such that the tubular member 20 is fluidly communicative with the container interior 33 (operation 52). Operations 51 and 52 are optional and may precede operation 50.

At this point, the user will close the second valve (operation 53) or confirm that the second valve is closed. Then, the user will actuate the first valve element 41 to place the first valve element 41 in the open condition to thereby admit fluid from the fluid source 410 into the tubular member 20 such that the fluid flows through the first tube end 21 and into the percutaneous cavity 24 (operation 54). The user will then actuate the first valve element 41 to place the first valve element 41 in the closed condition (operation 55) and will actuate the second valve element 42 to place the second valve element 42 in the open condition to thereby suction the fluid and fragmented particles from the percutaneous cavity 24, through the tubular member 20 and into the container interior 33 (operation 56).

As noted above, the fragmented particles will settle at the bottom of the container interior 33 and the fluid will either pool on top of the fragmented particles or the suctioning will continue such that at least some of the fluid is suctioned from the container interior 33 and through the straw 44. Operations 53-56 will then be repeated until all fragmented particles are removed from the percutaneous cavity 24. During each repetition, the various components of the device 10 may be rinsed or cleaned.

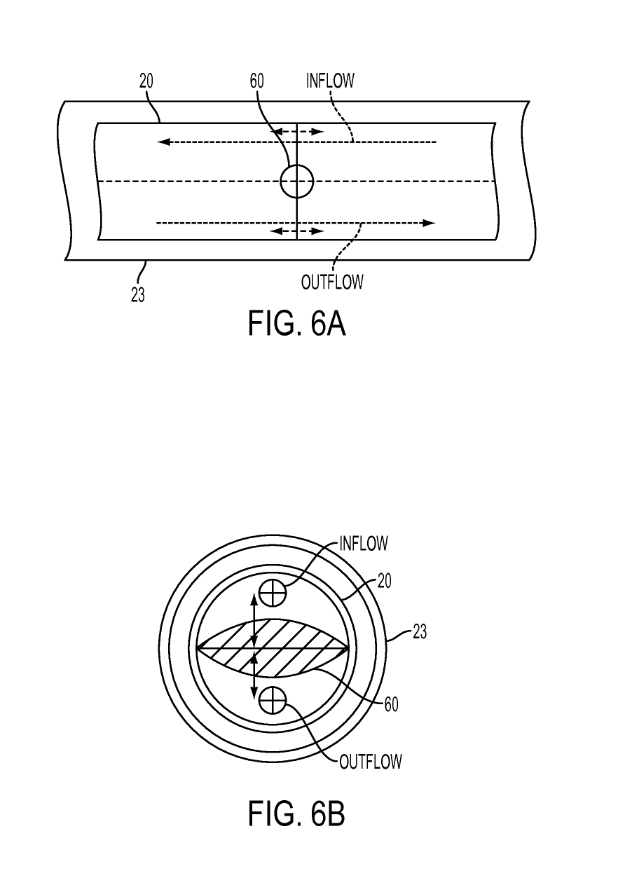

Although operations 53 and 54 and operations 55 and 56 are described above as being sequential, it will be understood that this is not required and that other embodiments are available. For example, the first and second valve elements 41 and 42 may be opened simultaneously such that fluid flow into the percutaneous cavity 24 occurs at a same time as the suctioning. In this case, the tubular member 20 could be divided hemishperically with one hemisphere used for inflows and one used for outflows. With this configuration, the pressure of the fluid ingress relative to the percutaneous cavity 24 in the inflow hemisphere will be substantially matched with the pressure of the egressing fluid in the outflow hemisphere such that fluid inflation of the percutaneous cavity 24 is avoided. With reference to FIGS. 6A and 6B, such pressure matching may be achieved by way of a two-way valve 60 that is coupled to both hemispheres to prevent inflows from exceeding outflows.

In accordance with further embodiments, it is to be understood that the device 10 is formed from generally inexpensive commonly available materials and that unit costs would be correspondingly limited. Thus, the various components of the device 10 could be disposable and replaceable during or following their use. For example, following a given PCNL procedure, the used tubular member 20 and the used first part 31 could be discarded and the used second part 32 (with the removed fragmented particles held therein) could be sent to a lab for analysis.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. A fluid supply and suction device, comprising:
a tube;
a container including first and second parts, which are sealably attachable to define a container interior, the first part being formed to define first and second apertures and being coupled with the tube such that the tube is fluidly communicative with the container interior; and
a valve system coupled with the first part and configured to admit via the first aperture fluid into the tube such that the fluid flows through the tube and to suction via the second aperture fluid and fragmented particles through the tube and into the container interior.

2. The fluid supply and suction device according to claim 1, wherein the first part comprises a body formed to define:
a receiving chamber sized to tightly fit around the tube; and
an inlet chamber coupled with the valve system, the inlet chamber being adjacent to the receiving chamber and proximate to the first aperture,
wherein an outer diameter of the inlet chamber is similar to an inner diameter of the tube.

3. The fluid supply and suction device according to claim 1, wherein the first and second parts are threadably engageable with each other.

4. The fluid supply and suction device according to claim 1, wherein the first and second parts are attachable to each other via mechanical or frictional interference.

5. The fluid supply and suction device according to claim 1, further comprising a seal element interposable between the first and second parts.

6. The fluid supply and suction device according to claim 1, wherein the first part comprises a baffle disposed to redirect outflow from the tube.

7. The fluid supply and suction device according to claim 1, wherein the valve system comprises:
a first valve element configured to selectively permit fluid flow into the tube;
a second valve element configured to selectively suction the fluid through the tube; and
a straw disposed to extend from the second aperture to a lower region of the container interior.

8. The fluid supply and suction device according to claim 7, wherein the first valve element is coupled to a fluid source and the second valve element is coupled to a vacuum source.

9. A fluid supply and suction device, comprising:
a tube having a first tube end, which is removably insertible into a sheath terminating in a percutaneous cavity, and a second tube end;
a container including first and second parts, which are sealably attachable to define a container interior, the first part being formed to define first and second apertures and being coupled with the second tube end such that the second tube end is fluidly communicative with the container interior; and
a valve system coupled with the first part and configured to admit via the first aperture fluid into the second tube end such that the fluid flows through the first tube end and into the percutaneous cavity and to suction via the second aperture the fluid and fragmented particles from the percutaneous cavity and into the container interior.

10. The fluid supply and suction device according to claim 9, wherein an inner diameter of the sheath is about 30 fr, an outer diameter of the tube is about 21-27 fr and an inner diameter of the tube is about 18-24 fr.

11. The fluid supply and suction device according to claim 9, wherein the fragmented particles are up to 4-7 mm in diameter.

12. The fluid supply and suction device according to claim 9, wherein the first part comprises a body formed to define:
a receiving chamber sized to tightly fit around the second tube end; and
an inlet chamber coupled with the valve system, the inlet chamber being adjacent to the receiving chamber and proximate to the first aperture,
wherein an outer diameter of the inlet chamber is similar to an inner diameter of the tube.

13. The fluid supply and suction device according to claim 9, wherein the first and second parts are threadably engageable to each other.

14. The fluid supply and suction device according to claim 9, wherein the first and second parts are attachable to each other via mechanical or frictional interference.

15. The fluid supply and suction device according to claim 9, further comprising a seal element interposable between the first and second parts.

16. The fluid supply and suction device according to claim 9, wherein the first part comprises a baffle disposed to redirect outflow from the second tube end.

17. The fluid supply and suction device according to claim 9, wherein the valve system comprises:
a first valve element configured to selectively permit fluid flow into the second tube end;
a second valve element configured to selectively suction the fluid and the fragmented particles through the tube; and
a straw disposed to extend from the second aperture to a lower region of the container interior.

18. The fluid supply and suction device according to claim 17, wherein the first valve element is coupled to a fluid source and the second valve element is coupled to a vacuum source.

19. A method of operating a fluid supply and suction device, comprising:
removably inserting a tube into a sheath terminating in a percutaneous cavity;
sealably attaching first and second parts of a container to define a container interior;
coupling the tube with the first part such that the tube is fluidly communicative with the container interior;
admitting fluid into the tube such that the fluid flows through the first tube end and into the percutaneous cavity; and
suctioning the fluid and fragmented particles from the percutaneous cavity and into the container interior.

20. The method according to claim 19, wherein the fragmented particles are up to 4-7 mm in diameter.
